(19) United States
(12) Patent Application Publication
(10) Pub. No.: US 2023/0086252 A1
(43) Pub. Date: Mar. 23, 2023

(54) METHODS AND SYSTEMS FOR OPERATING A LASER TO PERFORM FEMTOSECOND LASER ASSISTED CATARACT SURGERY

(71) Applicant: Patrick Sweeney, Plano, TX (US)

(72) Inventor: Patrick Sweeney, Plano, TX (US)

(21) Appl. No.: 17/951,820

(22) Filed: Sep. 23, 2022

Related U.S. Application Data
(60) Provisional application No. 63/247,374, filed on Sep. 23, 2021.

(51) Int. Cl. A61F 9/008

(52) U.S. Cl. CPC A61F 9/008; A61F 2009/0087; A61F 2009/00887

(57) ABSTRACT

This disclosure provides an improve methods and systems for performing Femtosecond Laser Assisted Cataract Surgery. By creating an inner radius in the eye capsule and lens fragmentation channels fluidically connected to the inner radius, then using viscoelastic fluid to separate the cataract, internal pressure is reduced on the peripheral portion of the cataract during the surgical procedure. Further, laser settings are oriented to permit instruments to break apart cataract nucleus into pieces with reduced ultrasound energy. The result of this improved method is a lessened likelihood of anterior capsular tears and a reduced chance of complications.

### FIELD

This application relates to the field of ophthalmology, and specifically, to femtosecond laser cataract surgical procedures.

### BACKGROUND

A cataract is a cloudy area in the lens of an eye that causes a decrease in vision. When a person is young, the person's eye lenses are able to accommodate effectively. However, as the person ages, the lenses harden, lose their elasticity, and lose their ability to accommodate in a physiological process known as presbyopia. Cataracts occur primarily as a result of this ageing process. Worldwide, cataracts are responsible for approximately 50% of blindness.

The only effective treatment is to surgically remove the lens with the cataract and replace it with an artificial intraocular lens. Cataract surgery is one of the most commonly performed surgeries in the United States. Cataract surgery typically involves phacoemulsification of the lens. Prior to performing phacoemulsification, a surgeon makes one or more incisions in the eye to allow the introduction of surgical instruments. In some surgeries, a surgeon manipulates the lens and breaks it into pieces. The surgeon inserts a phacoemulsification probe through an incision into the anterior chamber of the eye and emulsifies the lens, including the cataract, using pulsed ultrasonic vibrations. The lens is emulsified by the ultrasonic vibrations and aspirated out by suction.

Using a phacoemulsification probe to emulsify the lens is somewhat analogous to removing ice cream from a container using a spoon. If the ice cream is soft or semi-soft, scooping the ice cream with a spoon is relatively easy and minimal energy is required to remove the ice cream from the container. If the ice cream is hard, scooping the ice cream with a spoon is more difficult and more energy is required to remove the ice cream from the container. Similarly, when a lens is soft, manipulation and emulsification of the soft lens requires minimal energy and removal of the lens is relatively easy. However, when a lens is more hardened and less elastic, the surgeon must place a greater amount of torque upon the lens to manipulate and remove it. In such situations, the torque placed upon the lens can create a shearing force upon the structures that support the lens, including the capsular bag and zonules. Torque can cause capsular tears and zonular disruption. Further, a surgeon using additional torque on a hardened lens can inadvertently cause the tip of the phacoemulsification probe to pierce the capsular bag. Capsular tears, capsular piercings by phacoemulsification instruments, and zonular disruptions can cause vitreous prolapse. As a result of a vitreous prolapse, a patient may need a vitrectomy. The Centers for Medicare and Medicaid Services (CMS) has deemed vitrectomies as complications important enough to track. Thus, CMS instituted a tracking policy for unplanned anterior vitrectomies. Accordingly, surgical centers are required to report unplanned anterior vitrectomies. Currently, 1-2% of cataract surgeries result in unplanned anterior vitrectomies.

Femtosecond Laser Assisted Cataract Surgery (FLACS) is a surgical procedure that utilizes femtosecond laser technology to perform certain portions of the surgical procedure that would otherwise be performed manually. The femtosecond laser can be used to create an incision, a capsulotomy, and lens fragmentation, to make arcuate incisions to reduce astigmatism, or mark either the cornea or the capsule for astigmatism and intraocular lens implant positioning. After using a femtosecond laser, ultrasonic power is applied to break up and remove the cloudy natural lens.

Complications can still arise during FLACS procedures when a small capsulotomy is created or when excessive torque is applied to a dense cataract nucleus in a hardened lens. In these instances, radial anterior capsulotomy tears, capsular tears, and zonular dehiscence can still occur which may lead to complications including vitrectomy. In fact, although FLACS procedures have many benefits, including improving surgical accuracy, using less time and energy, and resulting in better visual outcomes, research has shown that FLACS procedures do not reduce the rate of vitrectomy. The average rate of vitrectomy remains around 1-2% for FLACS cataract surgeries. Accordingly, there is a need for an improved method of using a laser in Femtosecond Laser Assisted Cataract Surgery in a manner which minimizes the risk of radial anterior capsulotomy tears and decreases the complication rate.

### SUMMARY

In one aspect, this disclosure provides a method of operating a femtosecond laser to perform lens fragmentation for cataract surgery on the eye of a subject. The method includes positioning the laser so the laser beam of the laser aims or is aligned with a lens of the eye when the laser is operated. The method also comprises operating the laser to create an inner radius cut in a nucleus of the lens. The method comprises operating the laser to create an annular cut concentric to the inner radius cut. The method comprises operating the laser to create a plurality of lens fragmentation channels fluidically connected to the inner radius. The method comprises adding a viscoelastic fluid or balance salt solution to the lens. The method comprises removing the nucleus and removing the lens from the viscoelastic fluid.

In some embodiments, the laser is a femtosecond laser. In some embodiments, the plurality of lens fragmentation channels are offset within a range of 3 degrees to 8 degrees. In some embodiments, the laser is operated to make adjacent or overlapping radial cuts to create the lens fragmentation channels.

In some embodiments, removing the nucleus comprises applying torque to the nucleus.

In some embodiments, the viscoelastic fluid or balance salt solution separates or partially separates the lens into one or more pieces. Such separation makes for easier manipulation and removal by a surgeon.

In some embodiments, the method comprises configuring plane chop settings of the laser to comprise an outer radius of 4 millimeters, an anterior clearance of 1 millimeter, a posterior clearance value of about 0.55 to about 0.8 millimeter, and a rotate value of 57 degrees. In further embodiments, the method comprises configuring pie cut settings of the laser to comprise an outer radius of about 4 millimeters, a clearance anterior value of about 1 millimeter, a clearance posterior value of value of about 0.60 to about 0.75 millimeter, an inner radius value of about 0.35 to about 1.5 millimeter, a delta r value of about 1.5 millimeter, and a count value of 6. In still further embodiments, the method comprises configuring the laser to create the plurality of lens fragmentation channels at a specific diameter or width.

In another aspect, this disclosure provides a method of fragmenting a lens of an eye using a femtosecond laser. The method comprises positioning the femtosecond laser so that a laser beam of the laser is aligned with or aims at the lens when the laser is operated. The method comprises generating a laser beam comprising pulses and directing the laser beam to the lens. The method also comprises operating the laser beam on a central circular region of the lens, thereby creating a central circle. The method also comprises operating the laser beam in a circle concentric to the central circle, thereby creating a ring. The method comprises operating the laser beam to create a plurality of lens fragmentation channels fluidically connecting the central circle and the ring, wherein each lens fragmentation channel has a width sufficient to allow fluid flow and to fit a surgical instrument. The method comprises adding a viscoelastic fluid or balance salt solution to the eye.

In some embodiments of the method, the lens is fragmented into a plurality of pieces. Some embodiments further comprises separating and removing the plurality of pieces. In some embodiments, the method further comprises lifting and removing the central circle. Some embodiments additionally comprise inserting a cannula into the lens.

In some embodiments, the method further comprises operating the laser to create the plurality of lens fragmentation channels comprises operating the laser to make a plurality of overlapping or adjacent radial cuts resulting in a channel offset from about 3 to about 8 degrees. In some embodiments, the method further comprises operating the laser to create the plurality of lens fragmentation channels comprises making two or more overlapping incisions to create the channel.

A further aspect of this disclosure provides a system for performing lens fragmentation for cataract disassembly in the eye of a subject. The system comprises a laser source configured to produce a pulsed laser beam; an optical delivery system coupled to the laser source to receive and direct the pulsed laser beam; a processor coupled to the laser source and the optical delivery system, the processor comprising a tangible non-volatile computer readable medium comprising instructions to: direct the laser beam to fragment the lens of an eye using laser beam pulses to create: a first inner circle at the center of the eye; a first annular cut concentric to the first inner circle; and a plurality of lens fragmentation channels fluidically connected to the first inner radius and the second inner radius, wherein the lens fragmentation channels have a width sufficient to receive fluid and a surgical instrument. In some embodiments, the instructions further comprise instructions to direct the laser beam to make a plurality of overlapping or adjacent radial cuts to create the plurality of lens fragmentation channels by offset by about 3 degrees to about 8 degrees.

### BRIEF DESCRIPTION OF THE FIGURES

The invention and the following detailed description of certain embodiments thereof may be understood with reference to the following figure:

FIG. 1 is a flow diagram illustrating the improved method of removing a cataract using FLACS.

FIG. 2 is an illustrative drawing showing an inner radius and lens fragmentation channels on an eye capsule.

FIG. 3 is another illustrative drawing showing a perspective view of an eye capsule.

### DETAILED DESCRIPTION

The indefinite articles “a” and “an” mean one or more than one. The term “about” means ±10%. As used herein, the term “subject” means a mammal. In some embodiments, a subject is a human, dog, cat, horse, or monkey. Although reference is made herein to the LENSAR femtosecond laser, the methods disclosed herein are not limited to only the LENSAR laser and can be used with any femtosecond laser that can create the incisions and fluidic channels disclosed herein.

This disclosure provides systems and methods designed to improve the safety and efficiency of femtosecond laser assisted cataract surgery. In particular, it has been discovered that operating a laser in a particular fashion to fragment a patient's lens in a particular manner greatly reduces the risk of complications to the patient and the need for a vitrectomy. More specifically, operating a laser to perform lens fragmentation in the methods and systems disclosed herein results in an improved distribution of torque during manipulation and removal of the lens fragments. In some embodiments, the laser is used to create lens fragmentation channels of sufficient width to allow surgical instruments to pass through and viscoelastic fluid to pass through to separate the lens into pieces. The methods and systems described herein also result in a reduced amount of ultrasound energy required to fragment the cataract. Additionally, the addition of viscoelastic fluid to the lens fragmentation channels created by the methods disclosed herein allows for fluidic dissection. The risk of a posterior capsule rupture is mitigated by increasing the distance between the phacoemulsification tip and the posterior capsule. The methods and systems disclosed herein dramatically reduce the risk of complications by reducing the amount of torque needed to manipulate the cataract.

The method of this disclosure includes using a femtosecond laser to create an “inner circle” or “inner radius” in the capsule of the eye. The inner radius is a “circular button” in the center and is particularly helpful when the cataract nucleus is either too dense or the pupil diameter is too small so that a capsulorexhis would be too small. In some embodiments, the laser is also used to create one or more concentric ring cuts outside the inner radius. These additional concentric rings are concentric to and close to the inner radius, but not too close to the outer radius.

In cataract surgeries where the lens is unusually dense, or the capsulotomy is too small, extra torque and pressure are required to rotate and remove the lens pieces. This generates increased internal pressure on peripheral portion of the cataract nucleus/capsular bag complex, which in turn creates strain on the anterior capsule. The result is a greater likelihood of an anterior capsular tear.

In traditional cataract surgery or in FLACS surgery, instrumentation and torque must be delivered in the peripheral portion of the nucleus. This disclosure provides a new way to disperse and redistribute that torque to reduce the risk of a tear. The methods disclosed herein include using a laser to create an inner radius (also referred to herein as an “inner circle” or “central button”) in the eye capsule. In some embodiments of the method, the laser is used to create one or more ring cuts concentric to the inner radius. The laser is also used to create lens fragmentation channels in the eye capsule, wherein the lens fragmentation channels are fluidically connected to the inner radius and the concentric ring cuts, if made. The lens fragmentation channels are offset by about three to about eight degrees so that fluid can be passed to separate or partially separate the button into its own separate piece.

In some embodiments, the method comprises using the femtosecond laser to create channels in the lens that are offset by 3 to 8 degrees. In the method, for example, the laser is operated to make a radial cut from the center of the lens to the outer diameter. Then, the laser is operated to make overlapping or adjacent radial cuts to create a channel ranging from about 3 to about 8 degrees. In some embodiments, the outer diameter means the outer diameter setting on the laser. Offsetting the laser to make overlapping or adjacent cuts enables the creation of a channel of sufficient width so fluid can then be passed through the channel or a surgical instrument can be passed through the channel. The fluid or instrument can be used to separate or partially separate the lens. In some embodiments, the lens fragmentation channel has a width sufficient to fit a cannula. In certain embodiments, the lens fragmentation channel has a width sufficient to fit a standard 27 gauge Hydro dissection cannula. In some embodiments, the laser is used to make overlapping incisions to create the channels to create channels of sufficient width to allow fluid to pass through or a surgical instrument to pass through. In some embodiments, once this has been performed, the method comprises adding a viscoelastic material or balance salt solution to separate or partially separate the lens pieces from the center portion rather than placing torque in the peripheral portion of the nucleus. Placing torque in the peripheral portion of the nucleus places excessive strain on the anterior capsular opening. The anterior capsular opening can occasionally have some structural weaknesses, for example when a capsulotomy is smaller than 5.0 mm, or when an incomplete capsulorexhis is created, which is sometimes referred to as “postage-stamp type” perforations. If excessive force is placed in the periphery, and the strain is felt on the weaker anterior capsule opening, a radial anterior capsular tear can occur. This can essentially ruin attempts to remove the cataract and result in cataract pieces dropping into the posterior segment. This is a major complication. The methods disclosed herein significantly reduce the possibility of this complication. Additionally, when a capsulotomy is about 5.0 mm or smaller, particularly in situations with medium or severe cataract, there is an increased risk of an anterior capsular tear. Using the techniques described herein, separated segments can be lifted up and removed. In some embodiments, viscoelastic fluid or balance salt solution are used in the central button. The fluid lifts up the central button piece so it can be removed. The fluid can also be used to lift up the apical ends of the fragmented pieces and move them toward the center, while distributing torque under the anterior capsule is minimized.

When using the laser to create the inner radius, the distance between the outer radius and inner radius is referred to as “Delta R” or “ΔR.” In some embodiments of the method, the delta R value refers to the distance between the inner radius and one or more concentric circle cuts in the lens. In some embodiments of the method, the Delta R setting is used to make multiple concentric rings spaced apart at a certain distance. In some embodiments, the laser is used to create to one or more concentric ring cuts outside of the inner radius. In some embodiments, the laser is used to create 2 concentric ring cuts. In some embodiments, the laser is used to create 3 concentric ring cuts. In other embodiments, the laser is used to create 4 concentric ring cuts. In some embodiments, when the delta R is 0.75 mm, the laser is used to create an inner radius at 0.75 mm, a ring cut at 1.5 mm (i.e., 0.75 mm inner radius plus the delta R value of 0.75 mm), a ring cut at 2.25 mm, and a ring cut at 3 mm.

When setting values of a laser to perform the methods disclosed herein, some existing lasers only allow one thickness for all laser cuts to be performed. However, in some embodiments, the thickness of the laser beam used to create the inner radius or lens fragmentation channels is increased so that the inner radii cuts created by the laser are slightly wider. In some embodiments, the thickness of the laser beam is increased for the inner radius concentric circle cuts. In some embodiments, the increased thickness of the inner radius concentric circles can be sufficient to distribute torque for the particular cataract that is being operated on.

In some embodiments when the cataract is soft or when performing a refractive lens exchange, the inner radius setting can be 0.35 mm. In such embodiments, this inner radius setting results in the creation of a smaller volume of bubbles than with larger inner radius values. For surgeons who lack experience with a femtosecond laser, a smaller inner radius value, and smaller volume of bubbles, can be beneficial while learning the techniques disclosed herein.

In some embodiments, when performing cataract surgery with small capsulotomies, an inner radius setting of 0.75 mm is used to create an inner radius to allow for distribution of torque generated by the surgeon during the surgery.

In some embodiments, when performing cataract surgery on medium or hard cataracts, an inner radius setting of 0.75 mm is used to create an inner radius to allow for distribution of torque generated by the surgeon during the surgery. In some embodiments, when performing cataract surgery on hypermature cataracts or when the laser cannot identify the posterior capsule, an inner radius setting of 1.5 mm is used to create an inner radius to allow for distribution of torque generated by the surgeon during the surgery.

The viscoelastic material or balance salt solution lifting up the piece of the eye capsule created by the inner radius also helps separate and lift into the previously lasered channels, thereby making removal of the segments considerably easier.

This disclosure also provides systems for performing lens fragmentation for surgery. The system comprises a laser source configured to produce a pulsed laser beam. In some embodiments, the laser source is a femtosecond laser. In some embodiments, the system also comprises an optical delivery system coupled to the laser source to receive and direct the pulsed laser beam. In some embodiments, the optical delivery system comprises a lens. In some embodiments, the system comprises control electronics configured to control operation of the laser. In some embodiments, the control electronics comprise one or more processors coupled to the laser source and the optical delivery system. In some embodiments, the control electronics are communicatively coupled to a user interface. In some embodiments, the control electronics comprise tangible non-volatile computer readable medium configured to store instructions to be executed by the processor. In some embodiments, the instructions are configured to direct the laser beam to fragment the lens of an eye using laser beam pulses to create: a first inner circle at the center of the eye; a first annular cut concentric to the first inner circle; and a plurality of lens fragmentation channels fluidically connecting the first inner radius and the first annular cut, wherein the lens fragmentation channels have a width sufficient to receive fluid and a surgical instrument.

In some embodiments, the instructions further direct the laser beam to create the plurality of lens fragmentation channels by making a radial cut, then offsetting the laser to make adjacent or overlapping radial cuts to result in a channel of about 3 degrees to about 8 degrees wide. In some embodiments, the instructions further direct the laser beam to create overlapping incisions at specified angles to create the lens fragmentation channel.

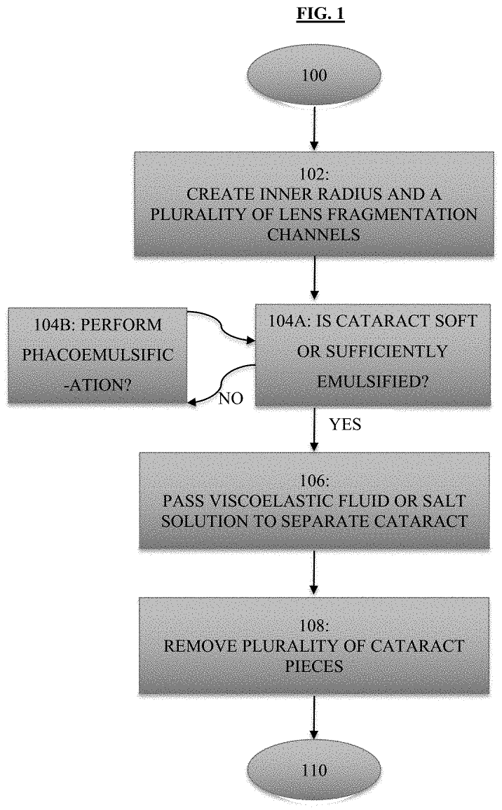

FIG. 1 is a flow diagram illustrating the improved method of removing a cataract using a laser during a FLACS procedure. Method 100 begins at step 102 using a femtosecond laser to create an inner radius in an eye capsule and creating a plurality of lens fragmentation channels. The lens fragmentation channels are offset within a range of 3 degrees to 8 degrees and are fluidically connected to the inner radius. In some embodiments, the method comprises operating the laser to create one or more ring cuts (not shown in FIG. 1) concentric to the inner circle and fluidically connected to the inner radius and lens fragmentation channels. Once the lens fragmentation channels are created at step 102, method 100 proceeds to step 104A where the cataract is evaluated to determine whether it is sufficiently soft or emulsified to pass a cannula to deliver viscoelastic fluid or balance salt solution in step 106. If the cataract is medium density or hard, then the method proceeds to step 104B where phacoemulsification is used to partially emulsify the cataract. A sufficient portion of the cataract is emulsified to allow the method to proceed to step 106. The depth of emulsification is at least approximate to the depth of the phacoemulsification tip. If the cataract is soft or sufficiently emulsified after phacoemulsification, then the method proceeds to step 106.

At step 106, method 100 comprises the step of passing viscoelastic fluid or balance salt solution to separate or partially separate the cataract into a plurality of pieces. Step 108 comprises removing the plurality of separate pieces of the cataract from the viscoelastic fluid or balance salt solution before method 100 terminates at step 110.

The software for operating the laser can be configured in certain values and settings to effectuate method 100. For example, when using a LENSAR® femtosecond laser in the method, the 3 plane chop settings of the laser can comprise an outer radius of 4 millimeters, an anterior clearance of 1 millimeter, a posterior clearance of about 0.55 to 0.8 millimeter, and a rotate value of 57 degrees. The pie cuts settings of the laser can comprise an outer radius of 4 millimeters, a clearance anterior value of 1 millimeter, a clearance posterior value of about 0.55 to 0.75 millimeter, an inner radius value of about 0.35 millimeter to about 1.5 millimeter, in some embodiments about 0.7 millimeter, in some embodiments 0.75 millimeter, in some embodiments 0.8 millimeter, a delta r value of 1.5 millimeter, and a count value of 6.

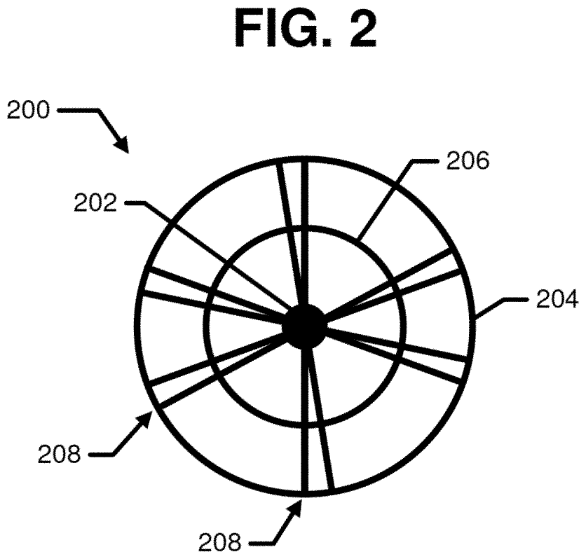

FIG. 2 is an illustration showing eye capsule 200. Inner radius 202 and outer radius 204 are shown. Inner radius 202 (also referred to herein as the “central button”) and inner radius 206 are shown. Delta R is the distance between inner radius 202 and inner radius 206. In some embodiments of the methods disclosed herein, the thickness of inner radius 202 and 206 can be varied to create a wider incision. Lens fragmentation channels 208 fluidically connected to inner radius 202 and inner radius 206. Lens fragmentation channels 208 have a width sufficient to receive a surgical instrument and a viscoelastic fluid or balance salt solution.

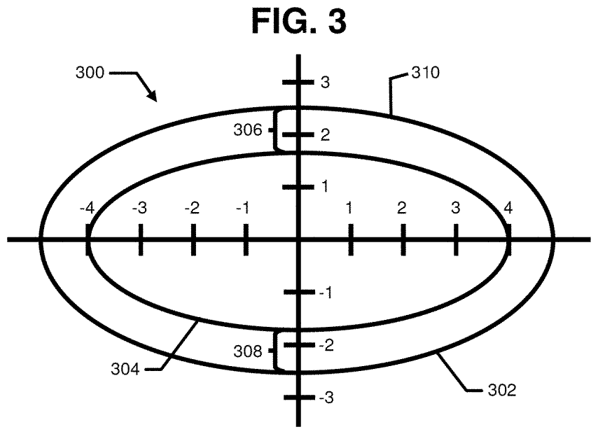

FIG. 3 is another illustrative drawing showing a sagittal perspective view of lens 300 and illustrating use of a laser in methods of this disclosure. 302 represents the posterior portion of the lens capsule. 304 represents the deepest depth of the lens fragmentation created by the laser in the methods disclosed herein. 308 represents the posterior clearance, which is the space between the posterior portion of lens capsule 302 and the most posterior portion of lens fragmentation depth 304 created by the laser. Posterior clearance 308 has a laser setting value of about 0.8 mm in FIG. 3.

310 represents the anterior (top portion) of the lens capsule. 306 represents the anterior clearance, which is the space between the anterior most portion of lens fragmentation and the top of portion of lens capsule 310. In FIG. 3, anterior clearance 306 has a laser setting value of about 1.0 mm. As shown in FIG. 3, the outer radius is 4 mm on the x-axis. In some embodiments of the methods disclosed herein, when using a LENSAR laser, the plane chop settings and pie chop settings of the laser can be configured as shown in FIG. 3 using the X and Y axis.

### EXAMPLES

### Example 1 Femtosecond Laser Assisted Cataract Surgeries

From 2021 until July 2022, 2,324 surgeries were performed on patients at Beltline Surgery Center in Sunnyvale, Texas. The surgical procedures involved two main steps: 1) femtosecond laser treatment and 2) cataract removal. First, the patient's name and date of birth were confirmed, then the eye slated for surgery was verified and the topography of the eye was verified. The patient's eye was anesthetized and ophthalmic draping was deployed to expose the eye slated for surgery.

Next, a femtosecond laser was aligned with the docking mechanism in the pre-operative area. The laser was directed to produce laser beam pulses at the eye. The laser beam was operated to create an opening in the lens capsule. Next, the laser engaged in lens fragmentation. The laser cut a central circle in the lens at a set diameter, cut an annular ring of set diameter concentric to the central circle, and cut a plurality of lens fragmentation channels by cutting overlapping radial laser cuts. The lens fragmentation channels were fluidically connecting the central circle and ring. Each lens fragmentation channel was cut by offsetting the laser from about 3 to about 8 degrees to create the channel. The lens fragmentation channels were cut from the inner radius to the outer radius setting on the laser.

Next, iris registration imaging and astigmatic arcuate incisions were made by the laser. After this portion of the procedure performed with the femtosecond laser, the patient was evaluated by an anesthesiologist and given appropriate medication for sedation. Patient was then taken to the operating room where proper anesthesia was maintained and the patient was prepped and draped in typical ophthalmic fashion. A sideport incision was made and viscoelastic fluid was added to the anterior chamber. A temporal near clear corneal incision was made and the lens capsule remnant was identified and remove.

Next, hydrodissection was performed with a flat tipped rectangular shaped , non-circular cannula or a curved tip cannula. The capsular bag was irrigated with Balanced Salt Solution (BSS) was used, just enough to allow rotation of the lens and cause the laser created air bubble to displace. Care was made to not use too much BSS in order to avoid excessive internal pressure in the bag (which can result in capsular tear, and subsequent complication of vitreous prolapse). In cases where the lens was reluctant to move, excessive force was avoided and additional BSS was used to fragment the lens in the laser fragmentation channels.

Subsequently, a flat tip hydrodissection cannula was turned 90 degrees sideways so the flat part (wide section of the rectangle shape) is against the pie-shaped segments created by the lens fragmentation channels. This allows the skinny part of cannula to slide in the lens fragmentation channel. In cases of hard cataracts, this was not possible since the cataractous lens is too hard to penetrate.

In cases of hard or severe cataracts where the lens cannot be penetrated due to density, phacoemulsification was used to shave the lens pieces down directly in the plurality of lens fragmentation channels to greater than 50% depth.

In the 2,324 femtosecond laser assisted cataract surgeries performed from 2021 until July 2022, using the laser to cut a central button, cut an annular ring concentric to the central button, and cut a plurality of lens fragmentation channels fluidically connected to the central button and the annular ring, only one patient required an anterior vitrectomy. This represents 0.04% of surgeries, a dramatic difference from the estimated 1-2% of surgeries that result in vitrectomies. Normal vitrectomy rates would have expected 23-46 patients requiring vitrectomies.

1. A method of operating a femtosecond laser to perform lens fragmentation for cataract surgery on the eye of a subject, the method comprising:
positioning the laser so the laser beam of the laser aims at a lens of the eye when the laser is operated;
operating the laser to create an inner radius cut in a nucleus of the lens;
operating the laser to create an annular cut concentric to the inner radius cut;
operating the laser to create a plurality of lens fragmentation channels fluidically connected to the inner radius;
adding a viscoelastic fluid or balance salt solution to the lens;
removing the nucleus; and
removing the lens from the viscoelastic fluid.

2. The method of claim 1, wherein the laser is a femtosecond laser.

3. The method of claim 1, wherein the plurality of lens fragmentation channels are offset within a range of 3 degrees to 8 degrees.

4. The method of claim 1, wherein removing the nucleus comprises applying torque to the nucleus.

5. The method of claim 1, wherein the viscoelastic fluid or balance salt solution separates or partially separates the lens into one or more pieces.

6. The method of claim 1, further comprising the step of configuring plane chop settings of the laser to comprise an outer radius of 4 millimeters, an anterior clearance of 1 millimeter, a posterior clearance value of about 0.55 to about 0.8 millimeter, and a rotate value of 57 degrees.

7. The method of claim 1, further comprising the steps of configuring pie cut settings of the laser to comprise an outer radius of about 4 millimeters, a clearance anterior value of about 1 millimeter, a clearance posterior value of value of about 0.60 to about 0.75 millimeter, an inner radius value of about 0.35 to about 1.5 millimeter, a delta r value of about 1.5 millimeter, and a count value of 6.

8. The method of claim 1, further comprising configuring the laser to create the plurality of lens fragmentation channels at a specific diameter or width.

9. A method of fragmenting a lens of an eye using a femtosecond laser, comprising
positioning the femtosecond laser so that a laser beam of the laser aims at the lens when the laser is operated;
generating a laser beam comprising pulses;
directing the laser beam to the lens;
operating the laser beam on a central circular region of the lens, thereby creating a central circle;
operating the laser beam in a circle concentric to the central circle, thereby creating a ring;
operating the laser beam to create a plurality of lens fragmentation channels fluidically connecting the central circle and the ring, wherein each lens fragmentation channel has a width sufficient to allow fluid flow and to fit a surgical instrument; and
adding a viscoelastic fluid or balance salt solution to the eye.

10. The method of claim 9, wherein the lens is fragmented into a plurality of pieces.

11. The method of claim 10, further comprising separating and removing the plurality of pieces.

12. The method of claim 9, further comprising lifting and removing the central circle.

13. The method of claim 12, further comprising inserting a cannula into the lens.

14. The method of claim 9, wherein operating the laser to create the plurality of lens fragmentation channels comprises operating the laser to make a plurality of overlapping or adjacent radial cuts resulting in a channel offset from about 3 to about 8 degrees.

15. The method of claim 9, wherein operating the laser to create the plurality of lens fragmentation channels comprises making two or more overlapping incisions to create the channel.

16. A system for performing lens fragmentation for cataract disassembly in the eye of a subject, comprising:
a laser source configured to produce a pulsed laser beam;
an optical delivery system coupled to the laser source to receive and direct the pulsed laser beam;
a processor coupled to the laser source and the optical delivery system, the processor comprising a tangible non-volatile computer readable medium comprising instructions to:
direct the laser beam to fragment the lens of an eye using laser beam pulses to create:
a first inner circle at the center of the eye;
a first annular cut concentric to the first inner circle; and
a plurality of lens fragmentation channels fluidically connected to the first inner radius and the second inner radius, wherein the lens fragmentation channels have a width sufficient to receive fluid and a surgical instrument.

17. The system of claim 16, wherein the instructions further direct the laser beam to make a plurality of overlapping or adjacent radial cuts to create the plurality of lens fragmentation channels by offset by about 3 degrees to about 8 degrees.
